(19) United States
(12) Patent Application Publication
(10) Pub. No.: US 2009/0297492 A1
(43) Pub. Date: Dec. 3, 2009

(54) Method for Improving Cognitive Performance

(75) Inventors: Akira SATOH, Shizuoka (JP); Shinji TSUJI, Shizuoka (JP)

Correspondence Address: McLELAND PATENT LAW OFFICE, P.L.L.C., 11320 RANDOM HILLS ROAD, SUITE 250, FAIRFAX, VA, 22030, US

(73) Assignee: YAMAHA HATSUDOKI KABUSHIKI KAISHA, Shizuoka (JP)

(21) Appl. No.: 12/475,010

(22) Filed: May 29, 2009

(30) Foreign Application Priority Data
May 30, 2008 (JP) 2008-142052
Apr. 21, 2009 (JP) 2009-102794
May 29, 2009 (JP) 2009-131448

(51) Int. Cl. A61K 38/43; A61K 36/16; A61K 36/8962; A61K 36/87; A61K 31/7088; A61K 31/122

(52) U.S. Cl. 424/94.1; 424/752; 424/754; 424/766; 514/44 R; 514/691

(57) ABSTRACT

The present invention provides a method for improving cognitive performance that encompasses all of: sense; perception; recognition; judgment; and action or suppression; and is based on a higher brain function. The method for improving cognitive performance of the present invention includes administering astaxanthin and/or an ester thereof to an individual. The cognitive performance is, for example, at least one selected from the group consisting of judging ability, spatial attention-allocating ability, concentration on the recognition of surroundings, and agility as determined by speed and/or accuracy of a body reaction which requires a higher brain function.

### BACKGROUND OF THE INVENTION

1. Field of the Invention

The present invention relates to a method for improving cognitive performance related to sport aptitude.

2. Description of the Related Art

In sports, appropriate body reactions must take place upon accurately perceiving, recognizing, and judging surrounding circumstances that change with time, such as instructions from a manager, the movements of the players on the opposing team and those of the players on one's own team, and, in the case of ball sports, the movements of a ball. For example, an ability called agility is determined by speed and/or accuracy of a body reaction which is based on cognitive functions. Accordingly, tests for checking agility such as tests for repetitive side steps at the sound of a whistle and tests for reaction to the flashing of a lamp are included in a battery of tests for checking the body functions of the aged or sports players. Thus, agility is an essential ability in athletics and sports.

Cognitive functions refer to higher brain functions that encompass: sense; perception; recognition; judgment; and action or suppression. More specifically, first, information on the surrounding circumstances is continuously transmitted via sense organs (visual sense, auditory sense, tactile sense, taste sense, and smell sense) to the brain. The brain has to select necessary information from among a large amount of information. At that time, the ability of a person to pay attention to a plurality of pieces of information (divided attention or allocated attention) is important. Examples of the plurality of pieces of information include: in the case of baseball, instructions from the bench, runners on base, opposing players in the field, and the pitching operations of a pitcher; and the like. Here, selected pieces of information are integrated to form one piece of information having particular meaning, that is, a perception. Next, perceived information is remembered for a short period of time, a current perception and a previous memory are checked against each other (recognition), and judgment is performed.

For example, when making a pass in a ball sport, agility, spatial attention-allocating ability, and other similar attributes are required in order to perform an action in which the direction, speed, timing, and other similar conditions for an operation are combined, based on the judgment to make a pass.

Such cognitive functions deeply relate to brain functions. There are a large number of various food components that are reported to improve brain functions. Examples of known components having an effect of improving brain functions include Ginkgo biloba leaf extract, DHA. (docosahexaenoic acid), L-carnitine, melatonin, coenzyme Q10, phospholipids, α-lipoic acid, nucleic acids, Ganoderma lucidum, ceramide, grape seed extract, polyphenols, pine bark extract, glucosamine, harp seal oil, Hericium erinaceum, Angelica keiskei, garlic extract, Cordyceps sinensis, turmeric, maca, cassis extract, theanine, (β-carotene, lycopene, and the like. Furthermore, the following has been reported regarding astaxanthin, which is a type of carotenoid.

For example, it has been disclosed that, regarding the visual sense, which is one of the senses, astaxanthin alleviates damage to the nerves in the brain, in particular, damage to nerves connected to the eyes and retina (U.S. Pat. No. 5,527,533). Furthermore, astaxanthin is known to have an effect in alleviating eye fatigue and eye controlling functional disorders of the eyes (WO 2002/094253). Thus, astaxanthin is considered generally to be good for alleviating eye strain. However, this action on the eyes is merely an effect on the function of an anatomically-based sense organ called the eye.

Furthermore, astaxanthin is known to have an effect relating to memory. For example, Japanese Laid-Open Patent Publication No. 2001-2569 discloses that a composition containing astaxanthin has an effect of preventing memory from deteriorating with age and an effect of improving memory after it has deteriorated. More specifically, in Japanese Laid-Open Patent Publication No. 2001-2569, tests on mice showed that astaxanthin had an effect on improving memory and learning ability and an action to improve memory. Furthermore, Japanese Laid-Open Patent Publication No. 2007-126455 discloses that a Haematococcus pluvialis extract containing astaxanthin has an effect of alleviating depression in a test on mice and an effect of alleviating functional disorders of the brain in a memory test on rats. Moreover, in view of the alleviation of functional disorders of the nerves in the brain, Japanese Laid-Open Patent Publication No. 2008-19242 discloses that astaxanthin can reduce mitochondrial dysfunction and oxidative stress on the nerve cells. However, all of these effects have been investigated only in animal models, and no conclusion may draw on the influence on higher brain functions in a human.

Moreover, regarding behavior, astaxanthin is known to have an effect on exercise. For example, it has been reported that astaxanthin can: improve the duration of muscle function, or treat muscle disorders or diseases (WO 99/11251); and can reduce and prevent oxidative stress generated dining exercise in animals (WO 2005/99478). Furthermore, Japanese Laid-Open Patent Publication No. 2007-314491 reports that the administration of astaxanthin increases creatine in the body without impairing kidney functions, thereby enhancing the athletic ability of muscles and other tissues. Moreover, Japanese Laid-Open Patent Publication No. 2006-347927 reports that astaxanthin has the effect of alleviating fatigue. However, all of these reports merely confirm the improvement of muscular strength and endurance and the reduction and prevention of damage to the muscle at the level of muscle tissues, and do not indicate an effect of improving a body's athletic ability as a result of improvement in the cognitive performance relating to higher brain functions.

Among carotenoids other than astaxanthin, there have been reports on the effects of β-carotene and lycopene on cognitive functions. It is merely known that lycopene can prevent damage to brain cells and cell death caused by active oxygen generated by ischemia-reperfusion during stroke or cerebral embolism (Kagome Co., Ltd. website, company information, news release, “Suggestion of possibility of protection of brain nerve cells by lycopene—joint study between Kagome Co., Ltd., College of Nagoya Bunri University, and Fujita Health University—”, Sep. 25, 2007 (accessed on May 28, 2008) on the Internet <URL: http://www.kagome.co.jp/news/2007/070925.html>). Furthermore, it has been reported that, when β-carotene was ingested for 18 years, cognitive functions such as verbal memory deteriorated less than in a placebo group which did not ingest β-carotene (Grodstein et al., Arch. Intern. Med., Vol. 167, pp. 2184-2190, 2007). However, these cognitive function tests (Grodstein et al., 2007) were limited to interview style dementia tests by telephone, in particular, verbal memory tests. Furthermore, there has been no report on the results from ingestion of β-carotene for approximately 1 year.

### SUMMARY OF THE INVENTION

It is an object of the present invention to provide an agent for improving cognitive performance when ingested for a short period, such cognitive performance encompassing all of sense; perception; recognition; judgment; and action or suppression; and being based on higher brain functions. It is also an object of the present invention to provide a method for improving cognitive performance.

The present invention provides an agent for improving cognitive performance, containing astaxanthin and/or an ester thereof.

In one embodiment, the cognitive performance is at least one selected from the group consisting of judging ability, spatial attention-allocating ability, concentration on recognition of surroundings, and agility determined by speed and/or accuracy of a body reaction which requires a higher brain function.

In one embodiment, the cognitive performance is evaluated by at least one of a neuropsychologic test and a neurophysiological test.

In a further embodiment, the test is at least one selected from the group consisting of tests for simple reaction, choice reaction, working memory, delayed recall, and divided attention.

In a particular embodiment, the test is for choice reaction, working memory, or divided attention.

In one embodiment, the cognitive performance is sport aptitude.

In one embodiment, the agent further contains at least one component selected from the group consisting of Ginkgo biloba leaf extract, unsaturated fatty acid, L-carnitine, melatonin, coenzyme Q10, phospholipids, α-lipoic acid, nucleic acid, Ganoderma lucidum, ceramide, grape seed extract, polyphenols, pine bark extract, glucosamine, harp seal oil, Hericium erinaceum, Angelica keiskei, garlic extract, Cordyceps sinensis, turmeric, maca, cassis extract, and theanine.

The present invention also provides a method for improving cognitive performance, the method comprising administering an effective amount of astaxanthin and/or an ester thereof to an individual.

In one embodiment, the method further comprises administering to the individual at least one component selected from the group consisting of Ginkgo biloba leaf extract, unsaturated fatty acids, L-carnitine, melatonin, coenzyme Q10, phospholipids, α-lipoic acid, nucleic acids, Ganoderma lucidum, ceramide, grape seed extract, polyphenols, pine bark extract, glucosamine, harp seal oil, Hericium erinaceum, Angelica keiskei, garlic extract, Cordyceps sinensis, turmeric, maca, cassis extract, and theanine.

The present invention provides a method for improving cognitive performance, based on the ingestion of astaxanthin for a short period, and specifically improving cognitive functions that are based on higher brain functions and encompass: sense; perception; recognition; judgment; and action or suppression. An object of this method for improving cognitive performance is not to alleviate or prevent functional disorders of the brain, such as so-called dementia or severe memory disorders, but to improve, for example, athletic aptitude, both of which depend on body functions, more specifically, higher brain functions. In sports requiring cognitive performance, the present invention is expected to result in improvement in a body's athletic ability.

### DESCRIPTION OF THE PREFERRED EMBODIMENTS

Astaxanthin and/or an ester thereof are contained as an active component in the agent used in the method for improving cognitive performance of the present invention. Astaxanthin and/or an ester thereof are a type of carotenoid represented by the following formula:

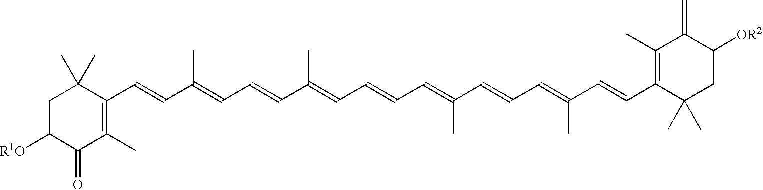

where R¹ and R² are both hydrogen in the case of astaxanthin, and R¹ and R² are each independently a hydrogen atom or a fatty acid residue provided that at least one of R¹ and R² is a fatty acid residue in the case of an ester of astaxanthin. Examples of the fatty acid residue in the ester of astaxanthin include, but are not particularly limited to, saturated fatty acids, such as palmitic acid and stearic acid, and unsaturated fatty acids, such as oleic acid, linoleic acid, α-linolenic acid, γ-linolenic acid, bishomo-γ-linolenic acid, arachidonic acid, eicosapentaenoic acid, and docosahexaenoic acid. The ester of astaxanthin can be any mono- or diester, homogeneous or non-homogeneous. Astaxanthin has a structure in which an additional oxo group and an additional hydroxy group are present at each end of a β-carotene molecule. Conversely, in an ester form (e.g., a krill extract) in which hydroxy groups at both ends are esterified with an unsaturated fatty acid or the like, the stability of the molecule is better.

Astaxanthin and/or an ester thereof used in the present invention may be chemically synthesized or derived from a naturally-occurring product. Examples of the naturally-occurring products in the latter case include: red yeast; the shell of crustaceans such as Tigriopus (red water flea) and krills; and microalgae such as green algae; which contain astaxanthin and/or an ester thereof. In the present invention, any extract containing astaxanthin and/or an ester thereof produced by any method may be used. Generally, extracts from the aforementioned naturally-occurring products can be used, and the extracts may be crude, or may be purified if necessary. In the present invention, a crude extract, a crushed powder of naturally-occurring products, a purified product thereof, and a product chemically synthesized, if necessary, that contain such astaxanthin and/or an ester thereof can be used either alone or in combination. In view of chemical stability, an ester form of astaxanthin is preferably used.

In the present invention, cognitive performance collectively refers to cognitive functions themselves, and abilities to exert such cognitive functions. Cognitive performance can be evaluated by various measuring methods, for example, neuropsychologic tests and neurophysiological tests. Examples of such a neuropsychologic test include CogHealth, Wechsler Adult Intelligence Scale, Stanford Binet Intelligence test, Visual Perception Test for Agnosia (VPTA), Standard Performance Test for Apraxia (SPTA), Wechsler Memory Scale-Revised, Clinical Assessment for Attention-Clinical Assessment for Spontaneity (CAT-CAS), Digit Cancellation Test for attention (D-CAT), Hamamatsu higher brain function scale, new Stroop test, Hasegawa dementia scale, Nishimura dementia scale, COGNISTAT, multiphasic early dementia examination (MEDE), NS dementia test, TAIS, MMSE, and the like. Examples of such a neurophysiological test include event-related potentials, and the event-related potentials include contingent negative variation (CNV), P1-N1-P2, NA, Nd, N2b, P300, MMN, N400, and the like. In a more medical sense, evaluation is possible also by measuring the activity of high-level functions of the brain using functional magnetic resonance imaging (fMRI), single photon emission computed tomography (SPECT), optical topography, or the like. Furthermore, as a popular method, evaluation is possible also with so called “brain training (Nou-Tore)”, which is a series of television game software. Moreover, such cognitive performance can be evaluated with, for example, measurements of physical fitness for checking the time taken to react to light.

CogHealth, mentioned above, is based on task-switching that is used for evaluating higher brain functions (executive functions), such as the suppression of an action or the reorganization of information processing (A. F. Kramer et al., Nature, Vol. 400, pp. 418-419 (1999), N. J. Cepeda et al, Developmental Psychology, Vol. 37, No. 5, pp. 715-730 (2001), and S. Hsieh and L. C. Liu, Brain Res. Cogn. Brain Res., Vol. 22, No. 2, pp. 165-175 (2005)). Furthermore, fMRI analysis of brain proves that frontal lobe activity and task-switching relate to each other, and thus CogHealth is accepted as a frontal lobe function test. CogHealth is a system in which the test subject is required to press a button in response to a card displayed on a monitor of a personal computer, for example, the system including five tasks (evaluation items) which are “simple reaction”, “choice reaction”, “working memory”, “delayed recall”, and “divided attention”. In CogHealth measurement, analysis is performed by calculating an average RT (reaction time) for each task.

“Simple reaction” refers to a task of pressing a button when a card is flipped from face-down to face-up. “Choice reaction” refers to a task of pressing a button based on the judgment of whether the card flipped from face-down to face-up is red or black. In other words, the simple reaction and the choice reaction are tasks relating to reaction time and suppression time in the function of the human frontal lobe. The simple reaction time is the time taken to perform one reaction to one sensory stimulus, and the choice reaction time is the time necessary to recognize a type of a given stimulus, and to make a reaction depending on the type in a predetermined manner.

“Working memory” refers to a task of pressing a button based on the judgment of whether or not a flipped card is the same as the preceding card. “Delayed recall” refers to a task of pressing a button based on the judgment of whether or not a flipped card is the same as a card that was present in the delayed recall task. “Working memory” and “delayed recall” are tasks for measuring memory, such as immediate memory and episodic memory. Working memory is a concept that includes not only information storage for a short period but also cognitive information processing. Working memory is memory used for a very short period of time in units of seconds, but the delayed recall is memory used for a period of time slightly longer than that of the working memory.

“Divided attention” refers to a task of pressing a button when at least one of five cards moving up and down is brought into contact with an upper or lower set line, and is a task for measuring spatially divided attention ability. In other words, such divided attention is an ability not to concentrate on one point but to allocate attention to the surroundings (spatially divided attention ability or spatially allocated attention ability). The meaning of divided attention is entirely different from attention scattering.

These tasks are not limited to card stimulation in CogHealth, but are tasks for evaluating higher brain functions employed also in tests using lamps or sound.

On the other hand, event-related potential, which is a type of electroencephalogram, is a method for measuring cognitive functions by measuring the activity of the cerebral nerves during cognitive work. In particular, the P300 (P3b) component of event-related potential has been used to estimate the mental work load (A. F. Kramer and T. Weber, Handbook of psychophysiology (2nd Edition), J. T. Cacioppo et al. (Eds.), New York, Cambridge University Press, (2000) pp. 794-814). In a state where two tasks are simultaneously executed, when one task is made difficult (or the priority of one task is increased), the execution performance of the other task is lowered. Based on a study using such a dual-task method, the P300 amplitude is considered to clearly reflect the cognitive functions in the perception-central nerve level that caused the amplitude (E. Donchin et al., Psychophysiology: Systems, processes, and applications, M. G. H. Coles et al. (Eds.), New York, Guilford Press, (1986) pp. 702-718). That is to say, the P300 amplitude reflects the degree of attention, the degree of concentration, and the degree of cognitive load, for a task of recognizing surroundings.

In a P300 test, for example, an auditory oddball paradigm can be employed. In this paradigm, two types of sounds consisting of a high-pitched sound having a frequency of 2 KHz and a low-pitched sound having a frequency of 1 KHz are emitted from a headphone, and a test subject is instructed to press a button as quickly and accurately as possible at the moment when the test subject hears the high-pitched sound. In this case, electrodes are arranged, for example, on the scalp at three points (Fz, Cz, and Pz) as defined in the International 10-20 System of Electrode Placement. After the electrodes are attached, during the test the test subject is instructed to gaze steadily at the fixed point of a “double circle” right in front of a chair on which the test subject is sitting. Then, a change in the obtained amplitude before and after ingestion is measured based on the Grand Average.

In human cognitive performance in daily life or sports, judging ability, spatially allocated attention ability, concentration on the recognition of surroundings, and agility determined by speed and/or accuracy of a body reaction, all of which require higher brain functions, are necessary. For example, when batting in baseball, a person needs to have the ability to concentrate on a ball that the pitcher throws while paying attention to a plurality of tasks, such as instructions from the bench and the movements of the players on the opposing team and those of the players on his/her own team on base, and to immediately swing a bat only when the ball comes into a course in which the bat is to be swung.

Herein, a sport refers to any sport, and is not particularly limited. Examples of a sport include outdoor sports, animal sports, combat sports and martial arts, target sports, water sports, gymnastics, team sports, and racket sports. Examples of outdoor sports include rafting, kite sports, and water sports (e.g., fishing, surfing, windsurfing). Examples of animal sports include equestrian arts and horse racing. Examples of combat sports and martial arts include sumo wrestling, judo, karate, jujutsu, Muay Thai, Shaolin temple kung fu, wrestling, mixed martial arts, boxing, kendo, and fencing. Examples of target sports include shooting, clay shooting, and golf. Examples of water sports include swimming, and water polo. Examples of gymnastics include gymnastic sports and rhythmic gymnastics. Examples of team sports include American football, soccer, futsal, water polo, softball, dodge ball, volleyball, handball, beach volleyball, football, field hockey, baseball, rugby, and lacrosse. Examples of racket sports include badminton, tennis, table tennis, and squash.

The agent for improving cognitive performance of the present invention may contain one or more optional component, as appropriate: a component that is considered to have an effect of improving cognitive functions, such as Ginkgo biloba leaf extract, unsaturated fatty acids (e.g., DHA, eicosapentaenoic acid (EPA), arachidonic acid, γ-linolenic acid), L-carnitine, melatonin, coenzyme Q10, phospholipids (e.g., phosphatidylcholine and phosphatidylserine), α-lipoic acid, nucleic acids, Ganoderma lucidum, ceramide, grape seed extract, polyphenols (e.g., resveratrol, curcumin, sesamin, and catechin), pine bark extract, glucosamine, harp seal oil, Hericium erinaceum, Angelica keiskei, garlic extract, Cordyceps sinensis, turmeric, maca, cassis extract, and theanine; a food material containing the component; or similar optional components.

The route of administration of the agent for improving cognitive performance of the present invention may be either oral or parenteral. The dosage form is selected appropriately according to the route of administration. Examples of the dosage form include parenteral solutions, infusion solutions, powders, granules, tablets, capsules, pills, enteric-coated preparations, troches, liquids for internal use, suspensions, emulsions, syrups, nose drops, ear drops, eye drops, inhalants, suppositories, and enteral nutrients. These can be used either alone or in combination. To prepare these dosage forms, auxiliary substances commonly used in the field of pharmaceutical manufacturing technology, such as excipients, binders, antiseptics, antioxidants, disintegrators, lubricants, and flavoring agents, can be used as necessary.

The dosage of the agent for improving cognitive performance of the present invention varies depending on the purpose of administration, the individual to whom it is administered (sex, age, body weight, etc.), and similar other factors. Typically, the dosage for an adult in terms of the free or unesterified form of astaxanthin may be 0.1 mg to 2 g per day, and preferably 4 mg to 500 mg per day, in the case of oral administration, while it may be 0.01 mg to 1 g per day, and preferably 0.1 mg to 500 mg per day, in the case of parenteral administration.

The agent for improving cognitive performance of the present invention can be used not only as a pharmaceutical as described above, but also as a product regulated as a “quasi-drug”, a functional food, a nutritional supplement, food and drink, and the like. When used as a quasi-drug, this agent may be used in conjunction with various auxiliary substances commonly used in the field of quasi-drugs or other technologies, if necessary. Alternatively, when used as a functional food, a nutritional supplement, or food and drink, this agent may be used in conjunction with additives commonly used for food products, such as sweeteners, spices, seasonings, antiseptics, preservatives, germicides, and antioxidants, if necessary. This agent may be used in any desired form such as a solution, a suspension, a syrup, granules, a cream, a paste, or a jelly, or may be shaped, if necessary. The ratio of the agent contained in these products is not particularly limited, and can be selected appropriately according to the mode of usage, and the amount of usage. In the present invention, it is preferably used as, for example, a functional food, a nutritional supplement, food and drink.

### EXAMPLES

### Preparation Example 1

### Preparation of Astaxanthin Capsules

First, astaxanthin was prepared in the following manner. Haematococcus pluvialis K0084 strain was cultivated at 25° C. under irradiation with light while bubbling a gas containing 3% CO₂ into the medium and under nutrient stress condition (i.e. nitrogen source deprivation), and then was encysted. The encysted cells were disrupted by a bead beater, and a lipophilic fraction containing astaxanthin was extracted with ethanol. The extract was concentrated under reduced pressure, and the ethanol was evaporated to give an extract containing astaxanthin in an amount of 8.0% expressed in terms of weight of the free form.

Soft capsules containing the components shown in Table 1 below per capsule were prepared using the extract containing astaxanthin in an amount of 8.0% expressed in terms of weight of the free form.

TABLE 1

| Component | Weight |
|---|---|
| Haematococcus extract (Yamaha Hatsudoki K.K.) | 52 mg |
| Olive oil (The Nisshin OilliO Group, Ltd.) | 78 mg |
| Vitamin E (The Nisshin OilliO Group, Ltd.) | 20 mg |

The obtained soft capsules contained astaxanthin in an amount of 3 mg per capsule expressed in terms of weight of the free form.

### Example 1

### Evaluation Test of Cognitive Functions with Administration of Astaxanthin

The test was performed on 10 healthy males aged 50 to 69 (age: 55.7±3.7) who had become aware of a tendency to age-related memory loss, but did not suffer from functional disorders of the brain, such as dementia. The astaxanthin-containing soft capsules (each capsule contains 3 mg of astaxanthin) were administered to the test subjects by ingestion of two capsules with water twice a day after eating breakfast and supper. The administration period was 12 weeks.

In this example, in order to evaluate cognitive function, measurement using CogHealth (based on task-switching) and P300 (one of the event-related potentials of an electroencephalogram) was performed at 6 weeks and 12 weeks after starting administration.

In CogHealth, the test subjects were instructed to press a button in response to a card displayed on a monitor of a personal computer. The average RT (reaction time) was calculated for each of the tasks “simple reaction”, “choice reaction”, “working memory”, “delayed recall”, and “divided attention”. For two memory tasks (“working memory” and “delayed recall”), analysis was performed by also calculating the average AR (correct answer ratio). The results are shown in Table 2 below.

In the electroencephalogram, an auditory oddball paradigm was used in which two types of sounds consisting of a high-pitched sound having a frequency of 2 KHz and a low-pitched sound having a frequency of 1 KHz were emitted from a headphone, and the test subjects were instructed to press a button as quickly and accurately as possible at the moment when the test subjects heard the high-pitched sound. Electrodes were arranged on the scalp at three points (Fz, Cz, and Pz) as defined in the International 10-20 System of Electrode Placement. The evoked electroencephalograms were measured with the frontal polar (Fpz) electrode as ground, and two linked electrode attached to the left and right earlobes as reference. An electrooculogram (EOG) was placed on the upper edge of the right or left eyehole. The contact impedance between the electrode and the scalp was set to 10 kΩ or less. If that was not possible, the contact impedance was set to 15 kΩ or less. After the electrodes were attached, during the test each of the test subjects was instructed to gaze steadily at a fixed point of a “double circle” right in front of a chair on which the test subject was sitting. Data with noise was taken as missing data, and the value of data in which P300 was above the base line was taken as 0. Measurement was performed twice, and the number of times of addition in each was set to 20. A change in the obtained latency and amplitude before and after ingestion was investigated based on the Grand Average. The results are shown in Table 3.

For each index, a multiple comparison (Dunnet) was performed to obtain the P value. Regarding the result of P300, a Student's t-test was used additionally as a comparison before and after ingestion.

TABLE 2

| | Before ingestion | After 6 weeks | | After 12 weeks | |
|---|---|---|---|---|---|
| Task | Mean ± SD | Mean ± SD | P value¹⁾ | Mean ± SD | P value¹⁾ |
| Simple reaction (msec) | 341.68 ± 94.41 | 303.31 ± 33.80 | 0.197 | 281.76 ± 33.56 | 0.034* |
| Choice reaction (msec) | 504.53 ± 56.84 | 480.63 ± 39.87 | 0.161 | 463.63 ± 26.49 | 0.013* |
| Working memory (msec) | 762.94 ± 141.65 | 732.95 ± 174.83 | 0.620 | 654.83 ± 128.42 | 0.014* |
| Delayed recall (msec) | 1008.19 ± 153.37 | 975.40 ± 190.75 | 0.553 | 916.77 ± 151.04 | 0.032* |
| Divided attention (msec) | 494.13 ± 135.57 | 419.52 ± 59.32 | 0.034* | 412.07 ± 51.97 | 0.020* |
| Working memory | 90.46 ± 7.18 | 9 5.22 ± 5.37 | 0.108 | 96.30 ± 3.94 | 0.045* |
| (Correct answer rate: %) | | | | | |
| Delayed recall | 70.95 ± 6.42 | 71.19 ± 5.98 | 0.991 | 70.71 ± 8.91 | 0.991 |
| (Correct answer rate: %) | | | | | |

¹⁾versus before ingenstion
*P < 0.05

In all tasks of CogHealth, a reduction in reaction time was confirmed. After the analysis of variance, post-hoc tests were performed. As a result, a significant reduction in the reaction time for divided attention was confirmed in a comparison between performances before ingestion and at 6 weeks after ingestion (P=0.034). In a comparison between performances before ingestion and at 12 weeks after ingestion, the reaction time was significantly reduced in all of the tasks: reaction time (P=0.034), choice reaction (P=0.013), working memory (P=0.014), delayed recall (P=0.032), and divided attention (P=0.020). Furthermore, regarding the correct answer ratio, a significant increase was confirmed in working memory (P=0.045), but no significant change was confirmed in the delayed recall (P=0.991).

TABLE 3

| | Before ingestion | After 12 weeks | |
|---|---|---|---|
| | Mean ± SD | Mean ± SD | P value¹⁾ |
| Amplitude (μV) | −7.60 ± 4.05 | −10.54 ± 3.39 | 0.057 |

¹⁾versus before ingenstion

In this example, a change in P300 at Fz was significant, and, thus, this was collected and analyzed. As a result of multiple comparisons, it was confirmed that no significant change was found in the amplitude, but a result close to a significant trend was obtained in a comparison of the amplitude before and after ingestion (P=0.057).

It was thus shown that, with the ingestion of astaxanthin, an effect of improving agility (reaction speed and reaction accuracy) as determined by speed and/or accuracy of a body reaction which requires higher brain functions, judging ability, and spatially allocated attention ability was confirmed by CogHealth, and an effect of improving concentration on the recognition of surroundings was confirmed by P300.

According to the present invention, it is confirmed by CogHealth that astaxanthin has an effect of improving agility (reaction speed and reaction accuracy) as determined by speed and/or accuracy of a body reaction which requires higher brain functions, improving judging ability, and improving spatially allocated attention ability, as well as an effect of improving concentration on the recognition of surroundings, as confirmed by P300. With these effects, the body's ability in daily life and during sports can be improved.

Astaxanthin and/or an ester thereof, which is contained in the agent for improving cognitive performance, has been consumed in food for a long time and this agent can be used not only as a pharmaceutical, but also as a health food product and the like used prophylactically on a daily basis.

What is claimed is:

1. A method for improving cognitive performance, comprising administering an effective amount of astaxanthin and/or an ester thereof to an indivisual.

2. The method of claim 1, wherein the cognitive performance is at least one selected from the group consisting of judging ability, spatial attention-allocating ability, concentration on recognition of surroundings, and agility as determined by speed and/or accuracy of a body reaction which requires a higher brain function.

3. The method of claim 2, wherein the cognitive performance is evaluated by at least one of a neuropsychologic test and a neurophysiological test.

4. The method of claim 3, wherein the test is at least one selected from the group consisting of tests for simple reaction, choice reaction, working memory, delayed recall, and divided attention.

5. The method of claim 4, wherein the test is for choice reaction, working memory, or divided attention.

6. The method of claim 3, wherein the cognitive performance is sport aptitude.

7. The method of claim 1, further comprising administering to the individual at least one component selected from the group consisting of Ginkgo biloba leaf extract, unsaturated fatty acids, L-carnitine, melatonin, coenzyme Q10, phospholipids, α-lipoic acid, nucleic acids, Ganoderma lucidum, ceramide, grape seed extract, polyphenols, pine bark extract, glucosamine, harp seal oil, Hericium erinaceum, Angelica keiskei, garlic extract, Cordyceps sinensis, turmeric, maca, cassis extract, and theanine.
